(19) United States
(12) Patent Application Publication
(10) Pub. No.: US 2007/0172888 A1
(43) Pub. Date: Jul. 26, 2007

(54) Measuring troponin antibodies to assess cardiovascular risk

(76) Inventors: Klaus Hallermayer, Feldafing (DE); Hugo Katus, Heddberg (DE); Ziya Kaya, Eppelheim (DE)

Correspondence Address: ROCHE DIAGNOSTICS OPERATIONS INC., 9115 Hague Road, Indianapolis, IN, 46250-0457, US

(21) Appl. No.: 11/340,012

(22) Filed: Jan. 25, 2006

(51) Int. Cl. G01N 33/53

(52) U.S. Cl. 435/7.1

(57) ABSTRACT

This invention relates to the field of myocardial disorders. It discloses that antibodies to a cardiac troponin found in a sample obtained from an individual can be used as a diagnostic marker, especially in the assessment of an individual's risk of developing a myocardial disorder. A method aiding in the assessment of an individual's risk of developing a myocardial disorder, comprising measuring in vitro antibodies to a cardiac troponin and optionally one or more other marker useful in assessing an individual's risk of developing a myocardial disorder, and correlating the value or the values obtained to the individual's risk of developing a myocardial disorder is decribed.

### FIELD OF THE INVENTION

The present invention relates to the field of myocardial disorders and the use of antibodies to a cardiac troponin found in a sample obtained from an individual as a diagnostic marker, especially in the assessment of the individual's risk of developing a myocardial disorder.

### BACKGROUND OF THE INVENTION

Despite significant advances in therapy, myocardial disease (CVD) remains the single most common cause of morbidity and mortality in the developed world. Thus, prevention of myocardial disorders such as myocardial infarction and stroke is an area of major public health importance. Several risk factors for future myocardial disorders have been described and are currently in wide clinical use in the detection of individuals at high risk. Such screening tests include for example evaluations of total cholesterol level, of LDL cholesterol level, of HDL cholesterol level and the level of C-reactive protein. However, a large number of myocardial disorders occur in individuals with apparently low to moderate risk profiles, and the diagnostic options to identify such patients is still limited.

Many cardiovascular complications will manifest themselves at the heart. These complications are summarized as coronary heart disease.

Individuals diagnosed as suffering from an underlying coronary heart disease can be divided into individuals showing no clinical symptoms and those which appear with breathlessness and/or chest pain. The latter group can be divided into individuals having stable angina pectoris (SAP) and those with acute coronary syndromes (ACS). ACS patients can show unstable angina pectoris (UAP), or these individuals have already suffered from a myocardial infarction (MI). MI can be a ST-elevated MI or a non ST-elevated MI. The occurring of a MI can be followed by a left ventricular dysfunction (LVD). LVD patients may undergo congestive heart failure (CHF) with a mortality rate of roughly 15%.

The heart is a unique organ. This is also true for the heart tissue and many of its constituents. The release of cardiac specific proteins into the circulation, e.g., as the result of a myocardial infarction, is a hall-mark of cardiac necrosis. The detection of such cardiac specific markers forms the basis of diagnostic means in the fields of myocardial infarction and congestive heart failure. It is well-known and established that an acute MI can be diagnosed with high sensitivity and at high specificity by measuring the level of a cardiac troponin in the circulation. Severe and acute stages of congestive heart failure can be diagnosed by measuring e.g. brain derived natriuretic peptide (BNP) or its N-terminal propeptide (NT-proBNP).

Whereas quite some progress has been made in the diagnosis of acute stages of myocardial complications a tremendous need still exists to further improve the diagnosis in acute stage settings, to differentiate between subsets of patients that may require different modes of treatment, and especially to establish and improve the assessment of an individual's risk of developing a myocardial disorder.

It has now been found that antibodies to a cardiac troponin can be used as a diagnostic marker, especially in the field of myocardial disorders. Antibodies to a cardiac troponin either alone or optionally in combination with one or more other marker of cardiovascular risk are valuable in the assessment of an individual's risk of developing a myocardial disorder.

### SUMMARY OF THE INVENTION

This invention describes new diagnostic tests that determine and utilize the presence, absence or level of antibodies to a cardiac troponin in the assessment of a myocardial disorder.

In one embodiment the present invention relates to method aiding in the assessment of an individual's risk of developing a myocardial disorder, comprising the steps of a) measuring in vitro antibodies to a cardiac troponin and optionally one or more other marker useful in assessing an individual's risk of developing a myocardial disorder, and b) correlating the value or the values obtained in (a) to the individual's risk of developing a myocardial disorder

These new tests broadly include (1) the assessment of risk of a future myocardial disorder such as for example myocardial infarction and congestive heart failure, and (2) the determination of the likelihood that certain individuals will benefit to a greater or lesser extent from the use of certain treatments designed to prevent and/or treat a myocardial disorder.

The present invention also discloses a kit comprising a cardiac troponin and auxiliary reagents appropriate for measurement of antibodies to said cardiac troponin.

These and other aspects of the invention will be described in more detail below in connection with the detailed description of the invention.

### DETAILED DESCRIPTION OF THE INVENTION

In a first preferred embodiment the present invention relates to the use of antibodies to a cardiac troponin as a diagnostic marker.

The skilled artisan is aware of different methods that may be used in the determination of antibodies as present in an individual's sample. The diagnostic field in which antibodies as present in a patient's sample are determined is called serology. The detection of an antibody comprised in a patient's sample is for example very important in the diagnosis of an infectious disease or of an autoimmune disease.

The release of a cardiac troponin is believed to occur only if cardiac tissue is damaged and becomes necrotic. Till the end of the 1990s the gold standard in detecting cardiac necrosis has been an elevated level of CK-MB (the cardiac-specific isoforms of creatinine kinase). At the end of the last decade cardiac troponins have emerged as at least as good a marker. The implications of troponin testing have been reviewed by Goldmann, B. U., et al., (Curr Control Trials Cardiovasc Med 2 (2001) 75-84). It is now generally accepted that a positive test for a cardiac troponin has a very high sensitivity in the detection of a myocardial infarction.

Dilated cardiomyopathy (DCM) is a myocardial disease characterized by progressive depression of myocardial contractile function and ventricular dilation. Recently Nishimura, H., et al. (Science 291 (2001) 319-322) reported that PD-1 receptor deficient mice develop severe DCM. They further found that these mice produce antibodies against cardiac troponin I.

It is known that antibodies to a cardiac troponin may be present in the circulation of some patients with acute coronary syndrome. These antibodies have been identified as the cause of discrepant data between different assays for measuring the same type of cardiac troponin. These anti-troponin antibodies lead to a decrease in assay sensitivity and thereby may cause a delay in the detection of a cardiac troponin (Eriksson, S., N. Engl. J. Med. 352 (2005) 98-100), e.g. after myocardial infarction.

The inventors of the present invention have now surprisingly found that the presence and/or level of antibodies against a cardiac troponin as determined in a patient's sample is of diagnostic utility. They can e.g. be used as a marker in the assessment of an individual's risk of developing a myocardial disorder.

The articles “a” and “an” are used herein to refer to one or to more than one (i.e. to at least one) of the grammatical object of the article. By way of example, “a marker” means one marker or more than one marker. In this invention “an antibody” and “antibodies” to a cardiac troponin is considered interchangeable, because, as the skilled artisan will appreciate it is always many antibodies which are detected.

Cardiac Troponins

A “cardiac troponin” is a troponin that is present in heart tissue and not present at all or not present to any relevant extend in tissue other than heart. By way of example for human beings two cardiac specific troponins have been described. These human cardiac specific troponins are known as troponin T, and I, respectively.

Troponin T has a molecular weight of about 37.000 Da. The troponin T isoform that is found in cardiac tissue (cTnT) is sufficiently divergent from skeletal muscle TnT to allow for the production of antibodies that distinguish both these TnT isoforms. TnT is considered a marker of acute myocardial damage; cf. Katus, H. A., et al., (J. Mol. Cell. Cardiol. 21 (1989) 1349-1353), Hamm, C. W., et al. (N. Engl. J. Med 327 (1992) 146-150), Ohman, E. M., et al. (N. Engl. J. Med. 335 (1996) 1333-1341), Christenson, R. H., et al., (Clin. Chem. 44 (1998) 494-501), and EP0394819.

Troponin I (TnI) is a 25 kDa inhibitory element of the troponin complex, found in muscle tissue. TnI binds to actin in the absence of Ca²⁺, inhibiting the ATPase activity of actomyosin. The TnI isoform that is found in cardiac tissue (cTnI) is 40% divergent from skeletal muscle TnI, allowing both isoforms to be immunologically distinguished. The normal plasma concentration of cTnI is <0.1 ng/ml (4 pM). cTnI is released into the bloodstream following cardiac cell death; thus, the plasma cTnI concentration is elevated in patients with acute myocardial infarction (Benamer, H., et al., Am. J. Cardiol. 82 (1998) 845-850).

In one preferred embodiment the present invention relates to a method aiding in the assessment of an individual's risk of developing a myocardial disorder, comprising: (a) measuring in vitro antibodies to a cardiac troponin and optionally one or more other marker useful in assessing an individual's risk of developing a myocardial disorder, and (b) correlating the value or the values obtained in (a) to the individual's risk of developing a myocardial disorder.

The method according to the present invention will “aid in the assessment” of an individual's risk of developing a myocardial disorder. As the skilled artisan will appreciate, no biochemical marker is diagnostic with 100% specificity and at the same time 100% sensitivity for a given disease. Rather, biochemical markers are used to assess with a certain likelihood or predictive value the presence, absence or severity of a disease. Therefore, in routine clinical diagnosis various clinical symptoms and biological markers are generally considered together in the diagnosis, treatment, and management of the underlying disease. The measurement of antibodies to a cardiac troponin will aid the physician in his task of establishing the correct diagnosis or prognosis. The final diagnosis is always made by the physician.

The terms “myocardial disorder” or “myocardial disorders” relate to a group of disorders affecting the heart muscle. A preferred group of myocardial disorders consists of arthroscleroses, congestive heart failure, acute coronary syndrome including myocardial infarction and unstable angina. Preferably the myocardial disorder assessed in a method according to the present invention is selected from the group consisting of congestive heart failure and acute coronary syndrome. Also preferred the term myocardial disorder in the sense of the present invention relates to the graft rejection in patients after heart transplantation.

The antibody to a cardiac troponin is measured “in vitro”. This means that a sample is obtained from an individual for diagnostic purposes. This sample is used for one or several in vitro investigations and not for treatment of said individual. Preferred samples are cardiac tissue biopsy, whole blood, plasma, or serum, especially preferred are plasma and serum.

In a preferred assay set-up for detection of an antibody to a cardiac troponin the cardiac troponin antigen is directly or indirectly bound to a solid phase. Usually the sample is diluted in a sample buffer. The solid phase bound antigen is incubated with the (diluted) sample under investigation. Incubation is performed under conditions permissive for binding of an antibody comprised in the sample under investigation to the solid phase bound antigen. The antibody attached to the solid phase bound antigen is detected by appropriate means.

In the detection of antibodies against pathogenic agents, such as viral pathogens, very frequently and to great advantage antibody detection systems according to the double antigen bridge format, e.g., described in U.S. Pat. No. 4,945,042, are used. The same assay principle can be used to detect antibodies to a cardiac troponin. The immunoassays according to this bridge concept require the use of an antigen directly or indirectly bound to a solid phase and of the same or a cross-reactive readily soluble antigen that is directly or indirectly detectable. The antibodies under investigation, if present, form a bridge between the solid phase bound antigen and the labeled detection antigen. Only if the two antigens are bridged by specific antibodies—e.g., by antibodies to a cardiac troponin—a signal is generated which is correlated to the concentration of antibodies present in the sample.

The cardiac troponin antigen used in a method according to the present invention in one preferred embodiment comprises troponin I and troponin T. It is also preferred to set up assays for the detection of antibodies to either troponin I, or troponin T. In the latter assays each cardiac troponin is individually used as an antigen. In a preferred embodiment the antibodies measured in a method according to the present invention are antibodies to troponin I.

In a preferred mode of performing the method according to the present invention a troponin from skeletal muscle is added to the sample buffer in order to enhance specificity of antibody binding to a cardiac troponin by blocking unspecific antibodies, i.e. antibodies cross-reacting between a muscle and a cardiac troponin.

As the skilled artisan will appreciate a test result may be recorded in qualitative and in quantitative terms.

The invention involves comparing the level of marker for the individual with a predetermined value. The predetermined value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as-a low-risk group, a medium-risk group and a high-risk group, or into quadrants, the lowest quadrant being individuals with the lowest risk and the highest quadrant being individuals with the highest risk.

The predetermined value can depend upon the particular population selected. For example, an apparently healthy population will have a different ‘normal’ range of markers than will a population the members of which have had a prior myocardial disorder. Accordingly, the predetermined values selected may take into account the category in which an individual falls. Appropriate ranges and categories can be selected with no more than routine experimentation by those of ordinary skill in the art.

A positive result may e. g. recorded if the antibodies measured are above a predetermined threshold level. Such threshold level usually is set to the 90%-percentile or to the 95%-percentile of a healthy control population. A threshold level at the 95%-percentile of a healthy control population is preferred when practicing this invention. Quantitative values need not to be explained to the man skilled in the art.

At present it is not known what causes the formation of antibodies to a cardiac troponin in an individual. It may be that a release of a cardiac troponin into the circulation occurs due to one or more necrotic events at the heart. The cardiac troponin in the circulation may trigger the formation of antibodies to said cardiac troponin.

The autoantibodies or briefly antibodies to a cardiac troponin may be of different immunoglobin classes.

In the course of an infection, first antibodies of the immunoglobin class M (IgM) are formed. The first humoral immune response in form of IgM is followed by a second humoral immune response, reflected by a more or less pronounced formation of antibodies of the immunoglobin class G (IgG). It is generally accepted the in the average the IgG-response will be the higher, the longer the “challenge” to the immune system lasts, e.g., in case of an infection, the longer the infection lasts and/or the more severe the infection is or has been, and/or the more often the infectious agent has triggered an immune response.

It has been found that the antibodies to a cardiac troponin as present in a patient's sample may comprise antibodies of the IgG as well as of the IgM class of immunoglobin. It may well be that different classes of antibodies to a cardiac troponin are indicative for different subsets of patients.

In a preferred embodiment the method according to the present invention is based on antibodies to a cardiac troponin that are of both the immunoglobin classes G, and M. A high titer in antibodies to a cardiac troponin may be considered indicative for a higher risk of further cardiac complications. In a further preferred embodiment the method according to the present invention is based on the

detection of antibodies to a cardiac troponin that are of immunoglobin class M (IgM). A high titer in IgM antibodies may be considered indicative for a more recent necrotic event at the heart muscle. A high titer of IgM antibodies may indicate a treatment more suited for acute events at the heart.

In another preferred embodiment the method according to the present invention is based on antibodies to a cardiac troponin that are of immunoglobin class G (IgG). A high titer in IgG antibodies may be considered indicative for at least one necrotic event at the heart muscle in the past. Such event in the past has most likely occurred at least four weeks before the sample has been obtained. A high titer of IgG antibodies may also indicate a severe and/or several necrotic episodes and may point to a mode of a treatment more suited for past and/or chronic events at the heart.

When an individual's sample has been analyzed with the method according to the present invention for his/her risk of suffering from a future myocardial disorder said individual can be stratified for one or more modes of therapeutic treatment. These can be selected from antibodies (monoclonal antibodies, polyclonal antibodies), small molecules, pharmacologically active compounds, i.e. anti-inflammatory and lipid-lowering drugs (e.g. statins), thrombolytic drugs (e.g. platelet antagonists), fibrinolytic drugs (e.g. heparin), revascularization therapy (e.g. PCTI (percutaneous therapeutic intervention), balloon dilatation, stenting, by-pass surgery).

Agents for reducing the risk of a myocardial disorder include those selected from the group consisting of anti-inflammatory agents, anti-thrombotic agents and/or fibrinolytic agents, anti-platelet agents, lipid reducing agents, direct thrombin inhibitors, and glycoprotein IIb/IIIa receptor inhibitors and agents that bind to cellular adhesion molecules and inhibit the ability of white blood cells to attach to such molecules (e.g. anti-cellular adhesion molecule antibodies).

Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone, Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant), rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide.

Lipid reducing agents include gemfibrozil, cholystyramine, colestipol, nicotinic acid, probucol lovastatin, fluvastatin, simvastatin, atorvastatin, pravastatin, cirivastatin.

Direct thrombin inhibitors include hirudin, hirugen, hirulog, agatroban, PPACK, thrombin aptamers.

Glycoprotein IIb/IIIa receptor Inhibitors are both antibodies and non-antibodies, and include but are not limited to ReoPro (abciximab), lamifiban, tirofiban.

One preferred agent which may be used to reduce the risk of a future cardiac disorder in an individual testing positive for antibodies to a cardiac troponin is aspirin. The method according to the present invention also may permit a therapeutic treatment

monitoring of the individual which is treated by said regimen.

In another surprising aspect of the invention, it has been discovered that antibodies to a cardiac troponin have a predictive value independent of other markers used in assessing an individual's risk of developing a myocardial disorder. In a further preferred embodiment the invention relates to a method aiding in the assessment of an individual's risk of developing a myocardial disorder, comprising: a) measuring in vitro antibodies to a cardiac troponin and one or more other marker useful in assessing an individual's risk of developing a myocardial disorder, and b) correlating the values obtained in (a) to the individual's risk of developing a myocardial disorder.

The one or more additional marker used together with an antibody to a cardiac troponin may be considered to be part of a marker panel for assessing an individual's risk of developing a myocardial disorder, i.e., a series of markers appropriate to further refine the risk assessment. The total number of markers in such marker panel is preferably less than 20 markers, more preferred less than 15 markers, also preferred are less than 10 markers, with 8 or less markers being even more preferred. Preferred are marker panels for assessing an individual's risk of developing a myocardial disorder comprising 2, 3, 4, 5, or 6 markers in addition an antibody to a cardiac troponin.

The skilled artisan is aware of a multitude of markers which have been described as useful assessing an individual's risk of developing a myocardial disorder. Preferably the one or more other marker will be selected from the group consisting of a cardiac troponin, a natriuretic peptide or a natriuretic peptide-related marker, an inflammation marker, D-dimer, cholesterol, homocysteine, adiponectin, sCD40L, myeloperoxidase, and ischemia modified albumin.

A “marker” is a molecule or feature whose absence, presence or level can be correlated to a status of interest, e.g., to a disease.

Natriuretic Peptides and Natriuretic Peptide-Related Markers

The natriuretic peptide preferably is selected from A-type natriuretic peptide (ANP) and/or B-type natriuretic peptide (BNP).

The term “related marker” as used herein refers to one or more fragments of a particular marker or its biosynthetic parent that may be detected as a surrogate for the marker itself or as independent markers. A natriuretic peptide related marker preferably is either an ANP-related or a BNP-related marker.

Brain Derive Natriuretic Peptide or B-Type Natriuretic Peptide (BNP)

Human BNP is derived by proteolysis of a 108 amino acid precursor molecule, referred to hereinafter as BNP₁₋₁₀₈. Mature BNP, or “the BNP natriuretic peptide,” or “BNP-32” or simply “BNP” is a 32 amino acid molecule representing amino acids 77-108 of this precursor, which may also be referred to as BNP₇₇₋₁₀₈. The remaining residues 1-76 of the BNP precursor molecule are known in the art as N-terminal proBNP (NT-proBNP).

BNP₁₋₁₀₈ is synthesized as part of an even larger precursor, the pre-pro-BNP, having 134 amino acids in total of which the N-terminal 26 represent the “pre-”sequence.

Mature BNP itself may be used as an additional marker in the present invention. The prepro-BNP, BNP₁₋₁₀₈ and NT-proBNP molecules all represent BNP-related markers that may be measured either as surrogates for mature BNP or as markers in and of themselves. In addition, one or more fragments of these molecules, including BNP-related polypeptides or markers selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP_{81- 108}, BNP₈₃₋₁₀₈, BNP₃₉₋₈₆, BNP₅₃₋₈₅, BNP₆₆₋₉₈, BNP₃₀₋₁₀₃, BNP₁₋₁₀₇, BNP₇₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. Pat. Ser. No. 10/419,059, filed Apr. 17, 2003, which is hereby incorporated by reference in its entirety.

In a similar fashion, many of the markers described herein below are synthesized as larger precursor molecules, which are then processed to provide the mature molecule or marker; and/or are present in circulation in the form of fragments and/or a marker molecule carrying secondary modifications. Thus, a “related markers” to each of the markers described herein below may be identified and used in an analogous fashion to that described above for BNP.

A-Type Natriuretic Peptide (ANP)

A-type natriuretic peptide (ANP) (also referred to as atrial natriuretic peptide or cardiodilatin Forssmann, W.-G., et al, Histochem. Cell Biol. 110 (1998) 335-357) is a 28 amino acid peptide that is synthesized, stored, and released by atrial myocytes in response to atrial distension, angiotensin II stimulation, endothelin, and sympathetic stimulation (beta-adrenoceptor mediated). ANP is synthesized as a precursor molecule (pro-ANP) that is converted to an active form, ANP, by proteolytic cleavage and also forming N-terminal ANP (1-98). N-terminal ANP and ANP have been reported to increase in patients exhibiting atrial fibrillation and heart failure (Rossi, A., et al., J. Am. Coll. Cardiol. 35 (2000) 1256-1262). As the skilled artisan will recognize, however, because of its relationship to ANP, the concentration of N-terminal ANP molecule can also provide diagnostic or prognostic information in patients. The phrases “marker related to ANP” or “ANP related peptide” refer to any polypeptide that originates from the pro-ANP molecule (1-126), other than the 28-amino acid ANP molecule itself. Proteolytic degradation of ANP and of peptides related to ANP have also been described in the literature and these naturally occurring proteolytic fragments are also encompassed it the term “ANP related peptides.”

Cardiac Troponin

The two cardiac specific troponins, i.e. troponin I, and troponin T, respectively, have been exemplified above. Whereas, above the use of a cardiac troponin as an antigen in the detection of anti-troponin antibodies is discussed, the cardiac troponin used as a further marker in a marker panel is the molecule itself.

One skilled in the art recognizes that in measuring a cardiac troponin, one can measure the different isoforms of troponin I and troponin T. Thus, one may preferably measure free cardiac troponin I, free cardiac troponin T, cardiac troponin I in a complex comprising one or both of troponin T and troponin C, cardiac troponin T in a complex comprising one or both of troponin I and troponin C, total cardiac troponin I (meaning free and complexed cardiac troponin I), and/or total cardiac troponin T. Preferably cardiac troponin I and/or cardiac troponin T are measured according to state of the art procedures and the values measured are combined with the result of a measurement for antibodies to a cardiac troponin and used in the assessment of a cardiac disorder. The presence of both of antibodies to a cardiac troponin and of a cardiac troponin may be further indicative for a recurring disease with acute coronary complications, like ACS.

If in an individual's sample elevated values are found for antibodies to a cardiac troponin as well as for a natriuretic peptide or for a natriuretic peptide-related marker this may be considered indicative of a situation of past myocardial damage which may already have become manifest as congestive heart failure and/or may represent an enhanced risk of future myocardial disease.

Preferred inflammation markers for use in a marker panel according to the present invention together with antibodies to a cardiac troponin are markers of acute inflammation and so-called proximal inflammatory markers.

Acute inflammatory markers known to the person skilled in the art include C-reactive protein (CRP), fibrinogen, D-dimer, serum amyloid A (SAA), pregnancy-associated polypeptide A (PAPP-A), intercellular adhesion molecules (e.g. ICAM-1, VCAM-1), IL-1-beta, IL-6, IL-18/IL-18b; TNF-alpha; myeloperoxidase (MPO); TF; monocyte chemoattractant protein 1 (MCP-1); P-selectin; E-selectin; platelet activating factor acetyl hydrolase (PAF-AH); von Willebrand Factor (vWF). Preferred markers of acute inflammation for use in a method according to the present invention are CRP, fibrinogen, D-dimer and SAA, of which CRP and D-dimer are more preferably used.

C-Reactive Protein (CRP)

C-reactive protein (CRP) is a homopentameric Ca²⁺-binding acute phase protein with 21 kDa subunits that is involved in host defense. CRP synthesis is induced by IL-6, and indirectly by IL-1, since IL-1 can trigger the synthesis of IL-6 by Kupffer cells in the hepatic sinusoids. The normal plasma concentration of CRP is <3 μg/ml (30 nM) in 90% of the healthy population, and <10 μg/ml (100 nM) in 99% of healthy individuals. Plasma CRP concentrations can, e.g. be measured by homogeneous assay formats or ELISA. C-reactive protein is considered a marker for ongoing systemic inflammation. Nowadays CRP can be measured with very high sensitivity and CRP-values in the range of between 1 and 3 mg/l of blood can be reliably detected. A measurement in that range is called a measurement of high-sensitive CRP or hs-CRP, which also is preferably used in a method according to the present invention.

Fibrinogen

Fibrinogen (also called Factor I) is a 340 kD protein encoded on chromosome 4 and synthesized by hepatocytes. It is composed of two identical subunits, each containing three dissimilar polypeptide chains (alphaA, betaB, gammaG) which are linked by disulphide bonds. Thrombin cleaves fibrinopeptides A and B from fibrinogen, resulting in the formation of strands of insoluble fibrin monomer which consists of three paired alpha, beta and gamma chains. Dysfibrinogenaemia is a condition associated with production of structurally abnormal fibrinogen. More than 250 structural variants have been described which are associated with a bleeding tendency (Ebert, R. F., CRC Press, Boca Raton, 1991). Most of these variants exhibit impaired thrombin-catalyzed release of fibrinopeptides, or impaired fibrin polymerization. Some variants of fibrinogen are associated with a thrombotic tendency rather than a bleeding tendency, and this has been attributed to impaired binding of plasminogen or tissue plasminogen activator to the abnormal fibrinogen molecule. Elevated levels of fibrinogen may be indicative for an ongoing infection or inflammation.

D-Dimer

D-dimer is a crosslinked fibrin degradation product with an approximate molecular mass of 200 kDa. The normal plasma concentration of D-dimer is <150 ng/ml (750 pM). The plasma concentration of D-dimer is elevated in patients with acute myocardial infarction and unstable angina, but not in stable angina. Hoffineister, H. M., et al., Circulation 91 (1995) 2520-2527. The plasma concentration of D-dimer also will be elevated during any condition associated with coagulation and fibrinolysis activation, including stroke, surgery, atherosclerosis, trauma, and thrombotic thrombocytopenic purpura. D-dimer is released into the bloodstream immediately following proteolytic clot dissolution by plasmin. The plasma concentration of D-dimer can exceed 2 μg/ml in patients with unstable angina. Gurfinkel, E., et al., Br. Heart J. 71 (1994) 151-155. Plasma D-dimer is a specific marker of fibrinolysis and indicates the presence of a prothrombotic state associated with acute myocardial infarction and unstable angina.

Proximal inflammatory markers are macromolecules situated upstream, i.e. close to or at the ethiopathogenetic origin of the disease event. In particular, they are produced at the site of the coronary heart lesion, preferably at the site of an arterial plaque. Proximal inflammatory markers are in particular associated with the risk that plaques already present in an individual will undergo inflammation, or growth, and with the probability of plaque rupture and thrombus formation.

Proximal inflammatory markers are known to the person skilled in the art, and non-limiting examples include pregnancy-associated polypeptide A (PAPP-A), matrix metalloproteinases (MMPs, e.g. MMP-1,-2,-3,-4,-5,-6,-7,-9,-10,-11, -12) and lipoprotein-associated phospholipase A2 (Lp-PLA2).

The preferred proximal inflammatory markers are PAPP-A, MMP-9 and Lp-PLA2. The most preferred proximal inflammatory markers are PAPP-A and Lp-PLA2, in particular PAPP-A.

Pregnancy-Associated Plasma Protein-A (PAPP-A)

The pregnancy-associated plasma protein-A (PAPP-A) belongs to the metzincin superfamily of zinc metalloproteinases. The molecular weight of PAPP-A is 187 kDa. Human pregnancy associated plasma protein A (PAPP-A) cleaves insulin-like growth factor (IGF) binding protein-4 (IGFBP-4), causing a dramatic reduction in its affinity for IGF-I and IGF-II. Through this mechanism, PAPP-A is a regulator of IGF bioactivity in several systems, including the human ovary and the cardiovascular system. A recent study shows that PAPP-A may also be a new candidate marker of acute coronary syndromes (Bayes-Genis, A., N. Engl. J. Med. 345 (2001) 1022-1029). The data in this study showed that PAPP-A levels are significantly elevated in patients with unstable angina or acute myocardial infarction when compared to patients with stable angina and control subjects.

Lipoprotein-Associated Phospholipase A₂ (Lp-PLA₂)

Lipoprotein-associated phospholipase A₂ (Lp-PLA₂) is a 50 kDa, Ca-insensitive lipase which is produced predominantly by macrophages. This enzyme resides mainly on low density lipoprotein (LDL) in human plasma. It is distinct from secretory phospholipase A₂ (sPLA₂). The levels of Lp-PLA₂ are not affected by acute systemic inflammatory conditions. Clinical studies have demonstrated that Lp-PLA₂ is related to atherosclerosis. Elevated plasma levels have been also found to correlate with CHD and ischemic stroke risk. In pre-clinical animal studies, inhibition of the enzyme attenuates the inflammatory process and slows down atherosclerotic disease progression.

Homocysteine

The concentration of circulating total homocysteine is a sensitive marker of inadequate folate and vitamin B 12 status. Elevated homocysteine concentrations are associated with an increased risk for vascular disease. Reference ranges (5th and 95th percentiles) for the total homocysteine concentration have been recently determined (Selhub, J., et al, Ann Intern Med. 131 (1999) 331-339). A high total homocysteine concentration was defined as one that exceeded the sex-specific 95th percentile for the reference sample. Reference ranges for serum total homocysteine concentration are age-dependent; these ranges are 4.3 to 9.9 micromole/L for male participants and 3.3 to 7.2 micromole/L for female participants 12 to 19 years of age and from 5.9 to 15.3 micromole/L for men and 4.9 to 11.6 micromole/L for women 60 years of age or older. A high homocysteine concentration was defined as at least 11.4 micromole/L for male participants and at least 10.4 micromole/L for female participants.

Adiponectin

Adiponectin is a protein of 226 amino acids which is produced mainly by adipocytes. The level of adiponectin appears to reflect insulin sensitivity and to link fat storage and arteriosclerosis. With regard to clinical utility several different intended uses are in discussion and/or under investigation. U.S. Pat. No. 6,461,821 describes and claims the use of adiponectin as a marker for arthrosclerosis.

Soluble CD40 Ligand (sCD40L)

The sCD40L has been supposed to be a marker of inflammation (Aukrust, P., et al., Circulation 100 (1999) 614-620) and hence to indicate a risk for the occurrence of coronary heart events. In WO 03/040691, sCD40L has been described as a systemic marker of inflammation. Recently sCD40L has also been discussed and described as a candidate marker for myocardial disorders. Heeschen, C., et al., N. Engl. J. Med. 348 (2003) 1104-1111, indicate that sCD40L might be used as a marker in acute coronary syndrome. sCD40L is in particular associated with platelet activation, platelet aggregation and thrombus propagation, representative of the risks that plaque having already become vulnerable will rupture, resulting in reversible vascular occlusion (UAP) or irreversible vascular occlusion (AMI) which may lead to left ventricular dysfunction (LVD), congestive heart failure (CHF) and death.

Cholesterol

Cholesterol at the same time is a steroid, a lipid, and an alcohol. It is found in the cell membranes of all body tissues, and transported in the blood plasma of all animals. Most cholesterol is not dietary in origin, it is synthesized internally. Cholesterol plays a central role in many biochemical processes, but is best known for its association with myocardial disease. Cholesterol travels through the blood in vesicles wherein it is attached to a protein. This cholesterol-protein package is called a lipoprotein. Lipoproteins are either high density or low density, depending on how much protein they have in relation to fat. Lipoproteins with more protein than fat are called high-density lipoproteins (HDL). Lipoproteins with more fat than protein are called low-density lipoproteins (LDL). High-density lipoprotein cholesterol is sometimes called “good” cholesterol. HDL cholesterol helps to remove LDL cholesterol from the body by binding with it in the bloodstream and carrying it back to the liver for disposal. A high level of HDL cholesterol appears to lower your risk of developing heart disease and stroke. Low-density lipoprotein cholesterol is sometimes called “bad” cholesterol. LDL cholesterol collects inside the walls of the arteries and often contributes to the plaque formation. LDL cholesterol is calculated from the total cholesterol, HDL, and triglyceride levels. In a further preferred embodiment of the present invention LDL cholesterol or the ratio of HDL to LDL is determined and used as part of a marker panel in order to assess an individual's risk of developing a myocardial disorder.

Myeloperoxidase

Myeloperoxidase is a lysosomal enzyme that is found in white blood cells, neutrophils. Myeloperoxidase is an enzyme that uses hydrogen peroxide to convert chloride to hypochlorous acid. The produced hypochlorous acid reacts with and destroys bacteria. In many inflammatory pathologies, such as cystic fibrosis and rheumatoid arthritis, neutrophils are also causing tissue damage. Myeloperoxidase is also produced when arteries are inflamed and have rupture-prone fatty deposits. An inflammation in the arteries can lead to a blood clot and eventually to a heart attack or stroke. Myeloperoxidase is considered a promising cardiac marker. By measuring the myeloperoxidase level in blood it is possible to predict whether a person is in risk of heart attack or death in the following six months (Baldus S., et al., Circulation 108 (2003) 1440-1445).

Placenta Growth Factor (PlGF)

Placenta growth factor (PlGF) is a polypeptide growth factor and a member of the platelet-derived growth factor family but more related to vascular endothelial growth factor (VEGF). PlGF-1 acts only as a very weak mitogen for some endothelial cell types and as a potent chemoattractant for monocytes. The physiological function in vivo is still controversy. In several reports it was shown not to be a potent mitogen for endotehlial cells and not angiogenic in vivo by using different assays. Very recently it was shown by one investigator, that PlGF-1 from cell culture supernatants was angiogenic in the CAM assay and in the rabbit cornea assay. Two different proteins can be generated by differential splicing of the human PlGF gene: PlGF-1 (131 aa native chain) and PlGF-2 (152 aa native chain). Both mitogens are secretable proteins, but PlGF-2 can bind to heparin with high affinity. PlGF-1 is a homodimer, but preparations of PlGF show some heterogeneity on SDS gels depending of the varying degrees of glycosylation. All dimeric forms posses a similar biological profile. There is good evidence that heterodimeric molecules between VEGF and PlGF exists and that they are biological active. A protein related of PlGF is VEGF with about 53% homology.

Ischemia Modified Albumin

The observation that myocardial ischemia produced a lower metal-binding capacity for cobalt to albumin (ischemia modified albumin or IMA) led to the development of the recently FDA-cleared albumin cobalt binding (ACB) test. The ACB test is a quantitative assay that measures ischemia-modified albumin (IMA) in human serum. In principle, cobalt added to serum does not bind to the NH2 terminus of IMA, leaving more free cobalt to react with dithiothreitol and form a darker color in samples from patients with ischemia. At present, the assay is available on a variety of clinical chemistry platforms. Specific preanalytical requirements need to be followed, including: avoiding use of collection tubes with chelators, performing assay analysis within 2.5 h or freezing at below −20° C., and avoiding sample dilutions. In addition, ACB test results should be interpreted with caution when serum albumin concentrations are <20 g/L or >55 g/L or in the presence of increased lactate or ammonia concentrations. Increased IMA values may be found in patients e.g. with cancer, infections, end-stage renal disease, liver disease, and brain ischemia. Several clinical studies have evaluated the performance of the ACB assay in cardiac patients, mostly examining the role of IMA in assessing ischemia. IMA may be considered as an additional marker to be included into a marker panel for assessment of an individual's risk of developing a myocardial disorder.

The method according to the present invention in a preferred embodiment is practiced in the investigation of apparently healthy individuals. “Apparently healthy”, as used herein, means individuals who have not previously had or at not aware of a previous adverse cardiovascular event such as a myocardial infarction. Apparently healthy individuals also do not otherwise exhibit symptoms of disease. In other words, such individuals, if examined by a medical professional, would be characterized as healthy and free of symptoms of disease.

As the skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated. This may e.g. be the case when diagnosing an infectious disease, like AIDS, by either detecting a nucleic acid or a polypeptide of the infectious agent or by detecting antibodies to the infectious agent. Frequently, however, the combination of markers is mathematically/statistically evaluated. Preferably the values measured for markers of a marker panel, e.g. an antibody to a cardiac troponin and the level of a cardiac troponin, are mathematically combined and the combined value is correlated to the underlying diagnostic question. Preferably the diagnostic question is the relative risk of developing a myocardial disorder in the future. Preferably the relative risk is given in comparison to healthy controls. Preferably healthy controls are matched for age and other covariates.

Marker values may be combined by any appropriate state of the art mathematical method. Well-known mathematical methods for correlating a marker combination to a disease or to the risk of developing a disease employ methods like, Discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention e.g. to the absence or presence of myocardial disease is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., J. of Computational and Graphical Statistics, 12 (2003) 475-511; Friedman, J. H., Regularized Discriminant Analysis, JASA 84 (1989) 165-175; Hastie, T., Tibshirani, R., Friedman, J., The Elements of Statistical Learning, Springer Series in Statistics, 2001; Breiman, L., Friedman, J. H., Olshen, R. A., Stone, C. J., (1984) Classification and regression trees, California: Wadsworth; Breiman, L. Random Forests, Machine Learning, 45 (2001) 5-32; Pepe, M. S., The Statistical Evaluation of Medical Tests for Classification and Prediction, Oxford Statistical Science Series, 28 (2003) and Duda, R. O., Hart, P. E., Stork, D. G., Pattern Classification, Wiley Interscience, 2nd Edition (2001).

It is a preferred embodiment of the invention to use an optimized multivariate cut-off for the underlying combination of biological markers and to e. g. discriminate patients with low, intermediate and high risk of developing a myocardial disorder. In this type of multivariate analysis the markers are no longer independent but form a marker panel.

Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease, respectively.

In each case, the ROC plot depicts the overlap between the two distributions by plotting the sensitivity versus 1−specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1−specificity [defined as (number of false-positive results)/(number of true-negative +number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left comer, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left comer to the upper right comer. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left comer, the higher the overall accuracy of the test.

One convenient goal to quantify the diagnostic accuracy of a laboratory test is to express its performance by a single number. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

In a preferred embodiment the present invention relates to a method for improving the assessment of an individual's risk of developing a myocardial disorder by measuring in a sample the concentration of an antibody to a cardiac troponin and the level of cardiac troponin and correlating the concentrations determined to the risk of developing a myocardial disorder.

In a preferred embodiment the present invention relates to a method for improving the assessment of an individual's risk of developing a myocardial disorder by measuring in a sample the concentration of an antibody to a cardiac troponin and the level of cholesterol and correlating the concentrations determined to the risk of developing a myocardial disorder.

In a preferred embodiment the present invention relates to a method for improving the assessment of an individual's risk of developing a myocardial disorder by measuring in a sample the concentration of at an antibody to a cardiac troponin and the level of CRP and correlating the concentrations determined to the risk of developing a myocardial disorder.

In a preferred embodiment the present invention relates to a method for improving the assessment of an individual's risk of developing a myocardial disorder by measuring in a sample the concentration of at an antibody to a cardiac troponin and the level of natriuretic peptide or a natriuretic peptide-related marker and correlating the concentrations determined to the risk of developing a myocardial disorder.

In preferred embodiments the invention provides novel kits or assays which are specific for, and have appropriate sensitivity with respect to antibodies to a cardiac troponin. A preferred kit accordingly to the present invention comprises a cardiac troponin and auxiliary reagents appropriate for measurement of antibodies to said cardiac troponin.

As discussed above the invention provides methods for evaluating the likelihood that an individual will benefit from treatment with an agent for reducing risk of a future myocardial disorder. This method may have important implications for patient treatment and also for clinical development of new therapeutics. Physicians select therapeutic regimens for patient treatment based upon the expected net benefit to the patient. The net benefit is derived from the risk to benefit ratio. The present invention may permit selection of individuals who are more likely to benefit by intervention, thereby aiding the physician in selecting a therapeutic regimen. This might include using drugs with a higher risk profile where the likelihood of expected benefit has increased. Likewise, clinical investigators desire to select for clinical trials a population with a high likelihood of obtaining a net benefit. The present invention can help clinical investigators select such individuals. It is expected that clinical investigators now will use the present invention for determining entry criteria for clinical trials.

The presence of an antibody to a cardiac troponin in an individual's sample may implicate that inflammatory processes are going on, which might lead to further damage of heart tissue. Anti-inflammatory therapy may be especially important for patients testing positive for antibodies to cardiac troponin I.

A cardiac troponin may be released into the circulation during a surgical intervention at the heart. This may be specially the case for patients undergoing surgery for heart transplantation. The release of a cardiac troponin during cardiac surgery may or may not trigger the formation of autoantibodies. Anti-troponin autoantibodies, however, once induced may well have a negative impact on the patient and may e.g. become relevant in rejection of the transplanted heart. In a further preferred embodiment, autoantibodies to a cardiac troponin will be of aid in the follow-up of patients after heart surgery, especially and preferably in the follow-up of heart transplantations.

Heart transplant patients that develop anti-troponin antibodies may require additional or different treatment as compared to patients not testing positive for such autoantibodies.

The following examples and references, are provided to aid the understanding of the present invention, the true scope of which is set forth in the appended claims. It is understood that modifications can be made in the procedures set forth without departing from the spirit of the invention.

### Specific Embodiments

### Example 1

### General Procedure for Detection of Antibodies to a Cardiac Troponin

In order to detect serum anti-cardiac troponin T, or troponin I antibodies the following assay can be used: Wells of a microtiter plate are first coated with a mouse monoclonal antibody to cardiac troponin T or I. In a second step the corresponding antigen, either cardiac troponin T or I is bound to the antibodies. By incubating an appropriately diluted serum sample with the bound troponin antigen the serum antibodies capable of binding to the troponin in the well will bind thereto. The bound serum antibodies can then be detected by an appropriate detection antibody, e.g. an anti-human IgG peroxidase conjugate. The skilled artisan is familiar with appropriate blocking and washing steps.

### Example 2

### Detection of Antibodies to Cardiac Troponin I in Human Serum Samples

Wells of a microtiter plate MaxiSorp® flat-bottom 96 well plate, Nunc order number 44-2404 were coated with an antibody to cardiac troponin I. Coating was performed at an antibody concentration of 0.5 μg/ml in coating buffer (=0.1 M NaHCO₃ Sigma order number S-51761, 34 mM Na₂CO₃; Sigma order number S-7795 pH 9.5) with 100 μl/well at 4° C. over night. Wells were washed thrice (300 μl per well and wash) with PBS/Tw (phosphate buffered saline NaCl Sigma order number S-5886, potassium chloride Sigma order number P-4504, sodium phosphate, Sigma order number S-5136, potassium phosphate monobasic, Sigma P-5655 with 0.05% Tween 20® Roth order number 9127.1). To block non-specific binding all wells received 300 μl of 1% gelatin (cold water fish skin, Sigma order no. G-7765) in PBS. Incubation was performed at RT for two hours. Wells were washed with PBS/Tw as above. Test wells received 100 μl of troponin I solution (3 μg/ml in sample diluent=PBS with 0.1% Tween 20® and 1% bovine serum albumin (BSA Sigma order number A-9647)). Control wells received 100 μl of sample diluent. Incubation was performed at room temperature (RT) for two hours. Wells were washed with PBS/Tw as above. Human sera were diluted 1:20 and further down in steps of 2 in sample diluent. Duplicates of 100 μl diluted serum per well were incubated at RT for 90 min in both test wells as well as control wells, respectively. Wells were washed with PBS/Tw as above. Per well 100 μl of detection antibody (Horseradish Peroxidase (HRP) conjugated anti-human IgG Monoclonal Antibody- BD Pharmingen product-no. 555788) diluted 1:10,000 in sample diluent were then added to each well and incubated for one hour, followed by washing as described above. Peroxidase activity bound to the wells was detected by use of 100 μl/well Blue Star TMB-HRP-Substrate (Diarect AG, product-no. DIA91000) as recommended by the supplier. Reaction was stopped after 45 min by adding 100 μl/well of 0.3 M H₂SO₄ J. T. Baker order number 6057. Extinction was recorded 450 nm using 550 nm as a reference wave length by SLT Spectra II, Tecan.

Median values were calculated for both the double determinations in test wells as well as for the double determinations in control wells. Corrected OD-results were calculated by subtracting the median of control wells from median of the corresponding test wells in order to compensate for non-specific binding. A corrected OD-value of more than 0.2 optical densities was considered as positive. Results are summarized in Table 1.

TABLE 1
Anti-troponin I antibody test results

| | Number of positive samples/ |
|---|---|
| Origin of samples | total number of samples |
| HOCM (hypertrophic obstructive | 5/7 |
| cardiomyopathy) | |
| DCM (dilated cardiomyopathy) | 6/13 |
| ICM (ischemic cardiomyopathy) | 5/13 |
| control (no known cardiac dysfunction) | 0/4 |

As can be seen, a significant number of patients suffering from cardiomyopathies of various kind has been found to have antibodies to cardiac troponin I in their sample. This is a clear indication that the presence of antibodies to a cardiac troponin may represent a hallmark of myocardial disease. Since (IgG) antibody production by the human body is not a matter of days theses antibody very likely may serve as a marker of risk for suffering from a cardiovascular disease, especially from a cardiomyopathy.

What is claimed is:

1. A method to aid in the assessment of an individual's risk of developing a myocardial disorder, the method comprising:
(a) measuring one or more in vitro antibodies to a cardiac troponin and
(b) correlating a value or values obtained in step (a) to the individual's risk of developing a myocardial disorder.

2. The method according to claim 1 wherein the cardiac troponin comprises troponin T and troponin I.

3. The method according to claim 1 wherein the cardiac troponin is troponin I or troponin T.

4. The method according to claim 1 wherein the antibodies are of the immunoglobin M class.

5. The method according to claim 1 wherein the antibodies are of the immunoglobin G class.

6. The method according to claim 1 wherein step (a) further comprises measuring a marker selected from the group consisting of a cardiac troponin, a natriuretic peptide or a natriuretic peptide-related marker, an inflammation marker, D-dimer, cholesterol, homocysteine, adiponectin, soluble CD40 ligand, myeloperoxidase, placenta growth factor, and ischemia modified albumin.

7. The method according to claim 6 wherein the marker is a cardiac troponin.

8. The method according to claim 6 wherein the marker is brain derived natriuretic peptide (BNP) or a BNP-related marker.

10. A kit comprising a cardiac troponin and auxiliary reagents appropriate for measurement of an antibody to the cardiac troponin.
